(19) United States
(12) Patent Application Publication
(10) Pub. No.: US 2010/0249542 A1
(43) Pub. Date: Sep. 30, 2010

(54) APPARATUS AND METHOD FOR DETECTION OF SYNCOPES

(75) Inventors: Jeroen Adrianus Johannes Thijs, Waldfeucht (DE); Jens Muehlsteff, Aachen (DE)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/746,242

(22) PCT Filed: Nov. 26, 2008
(86) PCT No.: PCT/IB2008/054958

§ 371 Date: Jun. 4, 2010

(30) Foreign Application Priority Data
Dec. 6, 2007 (EP) 07122515.5

(51) Int. Cl. A61B 5/02

(52) U.S. Cl. 600/301

(57) ABSTRACT

Apparatus for detecting impending vasovagal syncopes includes a plurality of ECG sensors (12), one of which is located close to the heart and a remote pulse sensor (22) located away from the heart. A processing unit (30) measures the pulse transit time for a pulse to travel from the heart ECG sensor to the remote pulse sensor.

### FIELD OF THE INVENTION

The invention relates to a method and apparatus for the detection of vasovagal syncopes.

### BACKGROUND OF THE INVENTION

A vasovagal syncope is a sudden loss of conciousness, the causes of which are not fully understood, but which involves a loss of blood pressure leading to fainting. In elderly patients, such syncopes are dangerous as fainting may lead to injuries from falls.

The population is aging and a large percentage of the population over seventy years of age will suffer a vasovagal syncope at some point over the next decade. One statistic suggests that 23% of the elderly population will suffer a syncope in ten years, and such events can account for 5% of emergency visits and 3% of hospital visits for such patients. The problems of vasovagal syncopes are accordingly widespread.

Such syncopes can represent a serious problem for elderly patients which makes it much more difficult for them to live independently.

It would be highly advantageous to provide apparatus to provide an advance warning of such a syncope.

A prior proposal is presented in US2007/0070800 which incorporates a photoplethysmographic sensor and estimates a probability of a syncope from the measured sensor signal.

An alternative solution is described in WO2002/41771 which measures blood pressure using a cuff.

### SUMMARY OF THE INVENTION

According to the invention there is provided apparatus according to claim 1.

By using the pulse transit time approach much more reliable results can be obtained than with the prior proposals mentioned above. The method is sufficiently simple that it can be incorporated into low power apparatus that can be permanently worn by the patient as the patient carries on their normal life.

Importantly, the approach adopted can give advance warning of an impending syncope and allow time for the patient to sit or lie down to minimise the risk of falling. In another aspect, the invention relates to a method according to claim 7.

### BRIEF DESCRIPTION OF THE DRAWINGS

For a better understanding of the invention embodiments will now be described with reference to the accompanying drawings in which:

FIG. 1 shows a patch;

FIG. 2 shows a wristband;

FIG. 3 shows a processing unit; and

FIG. 4 shows the steps of a method for detecting vasovagal syndrome.

The drawings are schematic and not to scale. Like components are given the same reference numerals in the different figures.

### DETAILED DESCRIPTION OF THE EMBODIMENTS

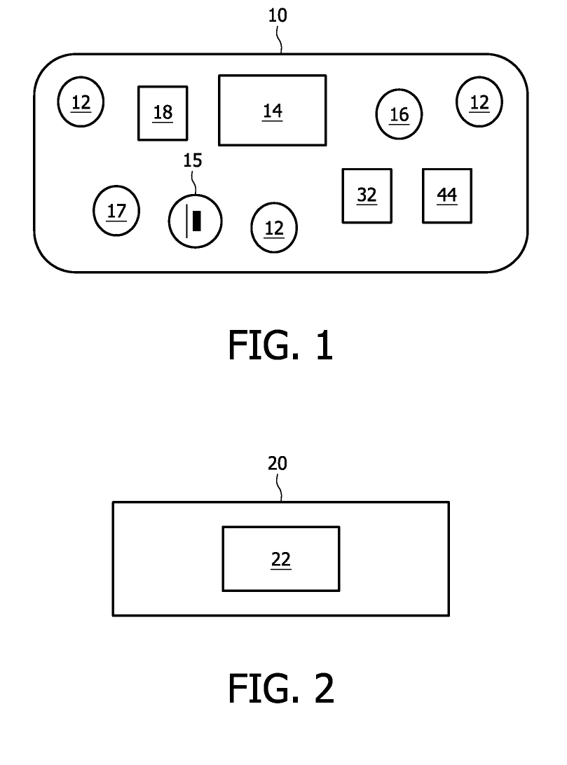

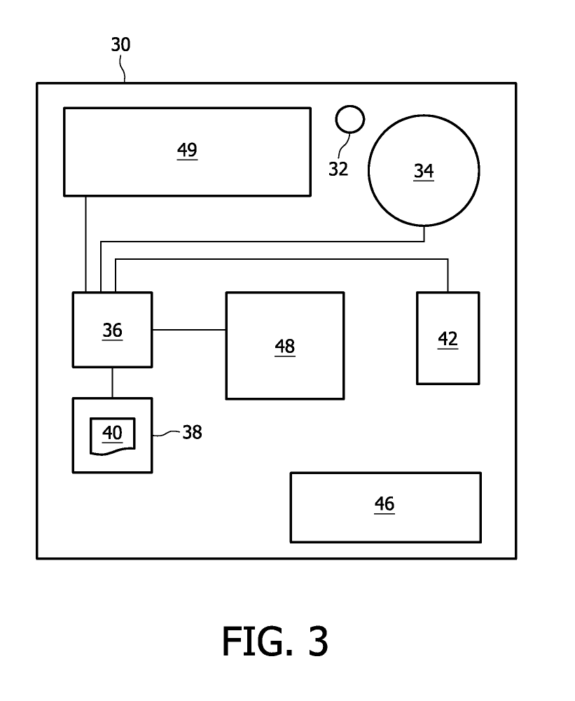

Referring to FIGS. 1 to 3, an embodiment of the invention includes a patch 10, a wristband 20 and a processing unit 30. The patch 10 is worn on the chest and in the embodiment is incorporated in an item of apparel, here a T-shirt.

A number of sensors are incorporated into the patch 10. The patch 10 includes ECG sensors 12.

The patch 10 also includes a further sensor, an accelerometer 14 for measuring activity and posture. A battery 17 is also provided, and the whole patch should use a very low power consumption. Other features of the patch are optional and are discussed below.

The wrist band 20 includes a pulse sensor 22.

The pulse sensor 22 may be a mechanical piezo sensor, for example, an optical sensor measuring a change in light absorption to detect a passing pulse wave, or a bioimpedance sensor.

A processing unit 30 is provided and worn on the body. The processing unit 30 includes a microphone 32 for capturing speech of the patient and a loudspeaker 34. The processing unit also includes a processor 36 and a memory 38 including code 40 for controlling the processor. A transceiver 42 is provided for providing radio contact and in particular for contacting the emergency services if required. A display 49 is provided to output data and operational messages.

The processing unit includes a battery 46, and operates at low power to achieve lengthy battery life without needing an excessive battery weight.

A memory 48 is used for recording sensor data over a period of time, in order to enable a retrospective analysis of a patient's vital signs. The memory 48 may store data over a period of 24 hours or more.

In use, the sensors continuously capture hemodynamic parameters and pass them to the processing unit 30. The parameters include ECG and pulse transit times. The data is processed and stored in memory 48.

Data, including for example ECG data, may be recorded over a period. This allows data taken during a syncope event. Such data may be of use to a medical practitioner caring for a patient since diagnosis of a vasovagal syncope is otherwise very difficult if the syncope is not observed by the medical practitioner.

Existing tests for detecting syncopes use complex computer systems and continual monitoring. However, it is important that the processing can be carried out on a low power unit. Thus, prior approaches which use complex data processing to carry out the check are not suitable. Since the existing tests are not suitable, the inventors have designed a simple algorithm that can be implemented in low power devices with low processing capabilities.

In particular, the inventors use a combination of the pulse transit time approach and context data. The context data include time, information regarding posture and activity measured from the accelerometer 44, as well as recorded speech and noises from the patient.

An important parameter is thus the pulse transit time which is measured by detecting the transit time of a pulse between the patch 10 and the wrist band 20. The pulse at the patch 10 may be determined from the ECG data, for example the R-peak in the ECG. The pulse at the wrist band may be measured by any convenient pulse detection technique.

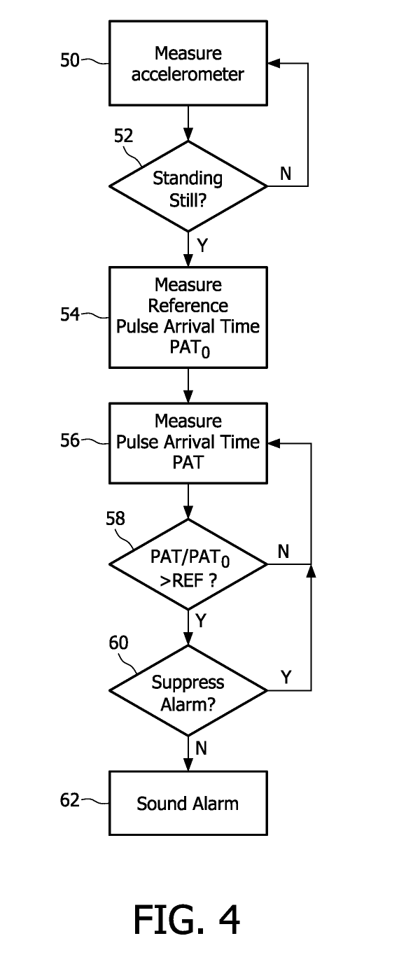

The simple algorithm used in the embodiment is as follows, as illustrated in FIG. 4. Firstly, the accelerometer is used (step 50) to detect posture change, and in particular to detect when the patient is stationary in a standing position (step 52). Then, as the patient stands still for a complete minute, the system measures a reference pulse transit time (PAT0), the time between a predetermined feature of the ECG pulse and the detection of the pulse at the pulse sensor (step 54).

Then, the system continues to monitor the pulse transit time in the same way (step 56). The ratio PAT/PAT0 of the measured pulse transit time (PAT) over the reference pulse transit time is calculated. When this ratio exceeds a predetermined limit (step 58), the context data is checked (step 60) to see if the patient is in a position or state for which the alarm is not to be sounded. In particular, the accelerometer data provides important context data. If the patient is relatively stationary and horizontal, i.e. asleep, there may be no need to sound the alarm which may accordingly be suppressed as unnecessary in this case.

If the ratio exceeds the limit, and the context data does not indicate that the alarm is unnecessary, the alarm sounds (step 62).

As will be appreciated, the code 40 is arranged to cause the processor 36 to carry out the steps of this method when the code is executed on the processor 36.

In an embodiment, the predetermined limit of the ratio PAT/PAT0 may be in the range 1.08 to 1.2.

This calculation is simple and hence does not overload the processing capability of the processing unit 30 yet is still capable of providing good advance warning for a vasovagal syncope.

The warning may take a number of forms. In the embodiment, a predetermined message is played on the loudspeaker. The message may be, for example, a message “lay down immediately! put your legs up”. The message may also be a warning message intended for passers by and bystanders.

After playing the warning message, the processor in the embodiment continues to monitor the patient. In the event of the patient not recovering quickly enough from the syncope, or in the case additional help is required, the processor calls the emergency services by automatically calling a call centre using the transceiver. In particular, if the patient has fallen this is picked up from the accelerometer data in which case the emergency services will be called.

In the embodiment, the ECG sensors 12 may include a plurality of capacitative sensors. These do not require good contact with the skin. Suitable sensors are disclosed in WO2007/060609 (Philips). The sensors are integrated into a textile item, here the patch of a T-shirt. As many sensors as are required may be used.

Alternative embodiments may accordingly include ten sensors in the standard ECG configuration, or a reduced number of sensors for example five sensors in the so-called EASI lead configuration which can then be used to calculate a derived ECG.

The sensors need not be integrated into a patch, but may also be integrated into a belt, or other item. Some sensors may be integrated into different wearable items. In a particular embodiment, a number of ECG sensors are integrated into a textile item for covering the thorax, for example a T-shirt. This provides a convenient wearable substrate for the ECG sensors.

The accelerometer 44 need not be incorporated into the patch, but may instead be incorporated into the textile item, belt, or even the processor 30 where worn.

The measurement and recordal of sensor data need not include only the factors mentioned above. Where additional data is recorded it may be used as context data for determining whether to sound or supress an alarm.

The patch 10 may include a temperature sensor 16, which may be used to record body temperature fluctuations. In cases where the equipment is used for the nighttime diagnosis of sleep problems, temperature may be a very relevant consideration.

Further, an impedance sensor 17 may be incorporated into the patch, which injects a small current at the thorax and hence measures the resistance. Impedance cardiography may be used to provide information about tissue composition of the thorax and the pump function and mechanical activity (stroke volume and cardiac output) of the heart.

A microphone 18 may be included in the patch. This can be used to measure heart and lung sounds. The microphone may be a piezo microphone. Heart and lung sounds give additional information about the heart and lung function, including valve sounds of the heart.

The accelerometer 44 may be a two axis or three axis accelerometer.

The system described has particular application to elderly care, cardiac rehabilitation and blood pressure Holter monitoring.

1. Apparatus for detecting impending vasovagal syncopes, comprising:
a plurality of wearable sensors (8) for arrangement on a human body, the sensors including a plurality of ECG sensors (12), one of the sensors being a heart sensor for arrangement in a vicinity of the heart when the sensors are arranged on the body;
a remote pulse sensor (22) for arrangement on the human body away from the heart; and
a processing unit (30) arranged to receive data from the ECG sensors (12) and the remote pulse sensors, to determine a pulse transit time for a pulse to travel from the heart ECG sensor to the remote pulse sensor, and to determine a presence or advent of a vasovagal syncope from the pulse transit time.

2. Apparatus according to claim 1, wherein the remote pulse sensor (22) is on a wristband (20).

3. Apparatus according to claim 1 wherein the heart sensor is one of the plurality of ECG sensors (12).

4. Apparatus according to claim 3 arranged to determine the pulse transit time as the time between a predetermined feature of the ECG measured using the heart sensor and the pulse being detected by the remote pulse sensor.

5. Apparatus according to claim 1 comprising a patch (10) on which the ECG sensors are arranged.

6. Apparatus according to claim 1 wherein the processing unit is arranged to determine the presence of a vasovagal syncope by comparing a ratio of the pulse transit time and a reference pulse transit time, and determining the presence of a vasovagal sycope when the ratio exceeds a predetermined value.

7. A method of processing data from a subject, comprising:
arranging a plurality of sensors over a body of the subject, the sensors including at least one heart sensor for arrangement in a vicinity of the heart and a remote pulse sensor (22) arranged on the human body away from the heart;
receiving data from the sensors;
determining a pulse transit time for a pulse to travel from the heart sensor to the remote pulse sensor; and
comparing a ratio of the pulse transit time and a reference pulse transit time.

8. A method according to claim 7, including
measuring the pulse transit time when the subject is standing still for a predetermined period;
using the measured pulse transit time as the reference pulse transit time.

9. A method according to claim 7, further comprising:
if the ratio of the pulse transit time and a reference pulse transit time exceeds a predetermined value, determining using an accelerometer if predetermined conditions exist.

10. A method according to claim 9, further comprising sounding an alarm if the ratio of the pulse transit time and a reference pulse transit time exceeds a predetermined value and the predetermined conditions exist.
